(19) United States
(12) Patent Application Publication
(10) Pub. No.: US 2023/0346199 A1
(43) Pub. Date: Nov. 2, 2023

(54) ANATOMY MEASUREMENT

(71) Applicant: 3Dintegrated ApS, Copenhagen N (DK)

(72) Inventors: Marco D.F. Kristensen, San Francisco, CA (US); Johan M.V. Bruun, Hellerup (DK); Mathias B. Stokholm, Soborg (DK); Job Van Dieten, Rodovre (DK); Sebastian H.N. Jensen, Copenhagen S (DK); Steen M. Hansen, Holte (DK); Henriette S. Kirkegaard, Copenhagen S (DK)

(21) Appl. No.: 17/733,358

(22) Filed: Apr. 29, 2022

(51) Int. Cl. G06T 7/60; A61B 1/00; G06T 19/00; G06T 7/73; G06T 3/40; A61B 1/313; G06T 11/20; G06T 7/55

(52) U.S. Cl. CPC A61B 1/0005; A61B 1/3132; G06T 3/4038; G06T 7/55; G06T 7/60; G06T 7/74; G06T 11/203; G06T 19/00; G06T 2200/24; G06T 2207/10068; G06T 2207/20101

(57) ABSTRACT

A surgical measuring system for minimally invasive surgery including a first image capture device configured to capture at least one first image of an interior of a cavity of a patient; a second image capture device configured to capture at least one second image of the interior of the cavity of the patient; at least one display; a processor; and a non-transitory computer readable medium storing instructions that cause the processor to perform a set of acts comprising: create, based on the at least one first image and the at least one second image, a depth map; display, on the at least one display, the at least one first image; determine, based on the depth map and the at least one first image, a distance between a plurality of specified points; and display, on the at least one display, the distance.

### BACKGROUND

Surgical systems may incorporate an imaging system, which may allow the clinician(s) to view the surgical site and/or one or more portions thereof on one or more displays such as a monitor. The display(s) may be local and/or remote to a surgical theater. An imaging system may include a scope with a camera that views the surgical site and transmits the view to a display that is viewable by the clinician. Scopes include, but are not limited to, laparoscopes, robotic laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes.

Surgical imaging systems may also involve stereo vision, which can allow for 3D reconstruction of patient anatomy captured by the imaging systems. Scene reconstruction or 3D reconstruction is a process of capturing the shape and appearance of real objects. Thus, allowing medical professionals to use a scope-based imaging system to capture, reconstruct, track, and potentially measure an internal area of a patient as well as any tools present in the images.

While various kinds of surgical instruments and image captures systems have been made and used, it is believed that no one prior to the inventor(s) has made or used the invention described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts an illustrative system for digital measurement;

FIGS. 2A and 2B depicts an interface which may be used to allow a user to select points and to display a cross section and three dimensional distance between them;

FIG. 3 depicts an interface showing a cross sectional view overlaid over an image of an interior of a cavity of a patient;

FIG. 4 depicts an interface showing a particular object with a highlighted resection margin;

FIG. 5 depicts an interface in which a three-dimensional (“3D”) representation is shown in a modal window on top of a surgical scene;

FIG. 6 depicts an example interface including tools and an indication of a distance between them;

FIG. 7 provides a schematic illustration of potential relationships between multiple data sources which could be used to generate a panoramic reconstruction;

FIG. 8 provides a schematic illustration of potential relationships between multiple data sources when keyframes are used to generate a panoramic reconstruction;

FIG. 9 provides a schematic illustration of potential relationships between multiple data sources when multiple keyframes are used in parallel to generate a panoramic reconstruction; and

FIG. 10 depicts a method which may be used to overlay a distance between points in three-dimensional space on a two dimensional image of an interior of a cavity of a patient.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose(s) described herein.

Similarly, the phrase “based on” should be understood as referring to a relationship in which one thing is determined at least in part by what it is specified as being “based on.” This includes, but is not limited to, relationships where one thing is exclusively determined by another, which relationships may be referred to using the phrase “exclusively based on.”

### I. System Overview

Disclosed herein are various systems and/or methods that relate generally to acquiring laparoscope stereo images and using image processing techniques to perform a digital measurement. The digital measurement may be based on a variety of factors, which will be discussed in greater detail herein.

The benefits of using minimally invasive surgery (MIS) are extensive and well known. Thus, improving the ability of surgeons to perform MIS and/or expanding the scope of procedures capable of being performed using MIS can lead to improved patient care.

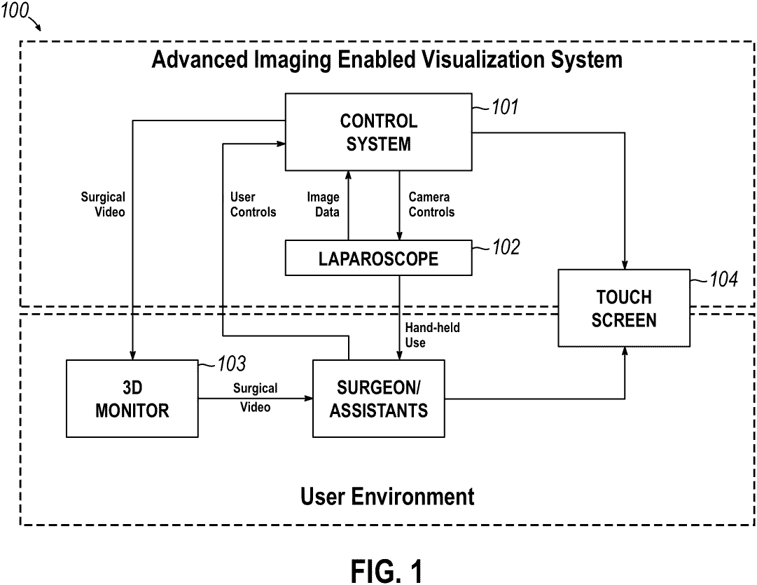

Referring now to FIG. 1, an illustrative system for digital measurement 100 is shown. In some embodiments, and as shown, the system 100 may comprise a control system 101, a stereo imaging device 102, such as a laparoscope, one or more three-dimensional (3D) monitors 103, and one or more user input devices, such as a 2D touch screen display 104. In some embodiments, the stereo imaging device 102 may capture at least two stereo images of a desired surgical scene that contains an object (e.g., an anatomical structure, surgical tool, etc.) to be measured. For example, a user (e.g., surgeon, first assistant, etc.) may navigate the stereo imaging device 102 to allow for viewing of a desired scene by watching the scene on a monitor (e.g., a surgeon monitor, which may be implemented using a 2D monitor, or a 3D monitor 103 as shown in FIG. 1, and/or a 2D monitor 104, in which either may be able to accept touch input), which may display images captured by the stereo imaging device in either 3D or 2D. It should be understood that although the stereo imaging device 102 allows for the creation of depth maps and 3D images, the surgical scene may not need to be reproduced or visualized in 3D when the user is generally viewing the surgical scene. For example, in some cases, either the surgeon monitor 103 and or a touch display 104 may display a two-dimensional image captured by a stereo imaging device (e.g., in a case where there are left and right image sensors, there may be displayed an image of the surgical scene captured by the left image sensor or the right image sensor, rather than a 3D reconstruction). As another example, in some cases, a two-dimensional image from the perspective of a virtual camera may be generated using a three-dimensional reconstruction and displayed on a surgeon monitor 103 and/or a touchscreen 104.

In some embodiments, once the stereo imaging device 102 captures at least two stereo images, those images may be sent to the control system 101. The control system 101 may then process the captured images. In one embodiment, the control system 101 may have one or more computer processing algorithms installed to allow for processing the pair of stereo images. In general, the captured images are considered to be red-green-blue (RGB) images, however, it should be understood that various other color spaces may be used, such as, for example, cylindrical-coordinate color models, color models that separate the luma (or lightness) from the chroma signals, and/or grayscale. In a further embodiment, the system may also utilize hyperspectral wavelengths for imaging.

Once two or more stereo images are captured by the stereo imaging device 102, the control system 101 may utilize one or more processing algorithms to derive depth information for the surgical scene. For example, a system such as shown in FIG. 1 may use a known distance between image capture devices to determine three dimensional locations for specific points (e.g., locations on tools, as described infra) through triangulation, or may create a disparity map, depth map or point cloud of the surgical scene. In support of deriving this type of three dimensional information, image sensors in a stereo image device may undergo a process of calibration to generate matrices and other data structures that would allow images captured by those devices to be rectified, have any distortions corrected, and be correlated with one another. In general the creation of the depth information may utilize any known or future method of 3D reconstruction, such as, for example projective reconstruction, affine reconstruction, Euclidean reconstruction, or the like. Generally, the difference between how the two image sensors perceive (e.g., see) the same scene can be used to create/calculate a depth map. Images and the resultant depth map may then be pre- and postprocessed to compensate for potential errors or issues (e.g., reflections, image noise, etc.), which can be prevalent in current laparoscopic images.

Once the depth information (e.g., a depth map) has been derived, the system may, in some embodiments, generate or construct a 3D representation of the surgical scene, including any objects that are present in the scene. For example, in some embodiments, there may be surgical tools present in the scene. In other embodiments, there may be specific patient anatomy (e.g., cysts, tumors, lesions, etc.). As will be discussed in greater detail herein, using the depth information and/or 3D reconstruction, the system can determine and/or measure distances between points in the surgical scene.

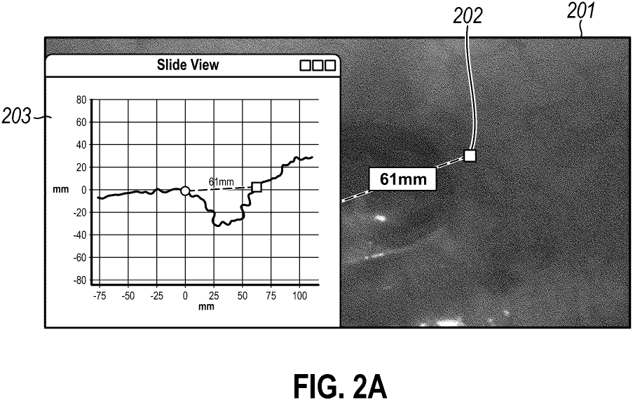

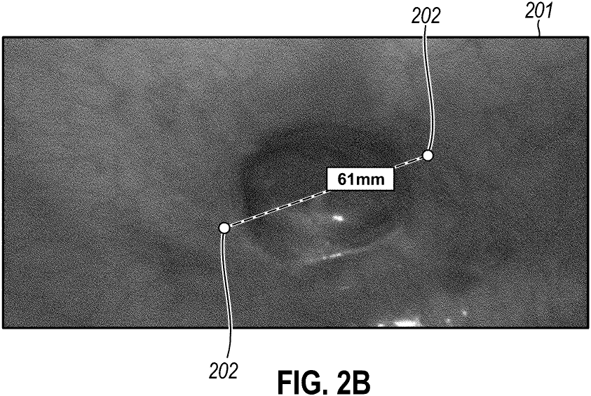

In some embodiments, and as shown in FIG. 1, some operating rooms (“OR”) may have one or more display devices (e.g., the 3D monitor 103, a 2D monitor/touch screen 104, a wearable/head mounted display (not shown), and the like). Utilizing one or more of the display devices 103/104, the system may present a 2D or 3D image to the surgeon or surgical team. Referring now to FIGS. 2A and 2B, an illustrative example of a display is shown. In some embodiments, and as shown, the display device 103/104 may display the surgical scene 201. Two or more points 202 may be selected, such as by the user touching those points or dragging his or her finger across them on a touch screen display displaying a two-dimensional image of the surgical site. Using these points, as well as depth information for the surgical site, the system can calculate and display a measured distance between the two or more points within the surgical scene.

As will be discussed herein, various methods exist for determining the location of the plurality of specified points 202. Stated differently, systems and methods, as disclosed herein, may be capable of receiving inputs from various input-output (I/O) devices, such as, for example, a touch screen, keyboard, mouse, gesture recognition, audio recording, and the like. By way of non-limiting example, the system may, in some embodiments, display one of the stereo images of the surgical scene 201 (e.g., the left image or right image) on a touchscreen device 104, while in other embodiments, the system may display a 3D reconstruction image of the surgical scene 201 on a touchscreen device 104. A user may then use their finger or a stylus to provide user input to the interface (e.g., by dragging the stylus across the two-dimensional image shown on a touch display) in order to select and/or specify two or more points 202 to be measured. These measurements may then be displayed on the image on which they were specified (e.g., a snapshot of a surgical scene), or may be displayed on real time images of a surgical scene during a procedure (e.g., locations of points may be tracked over time, and data such as Euclidian distances between points may be overlaid on a display which is updated with new information regarding a surgical scene as it is available). In a further embodiment, the user may also perform additional actions, such as, navigating and/or drawing on the displayed image.

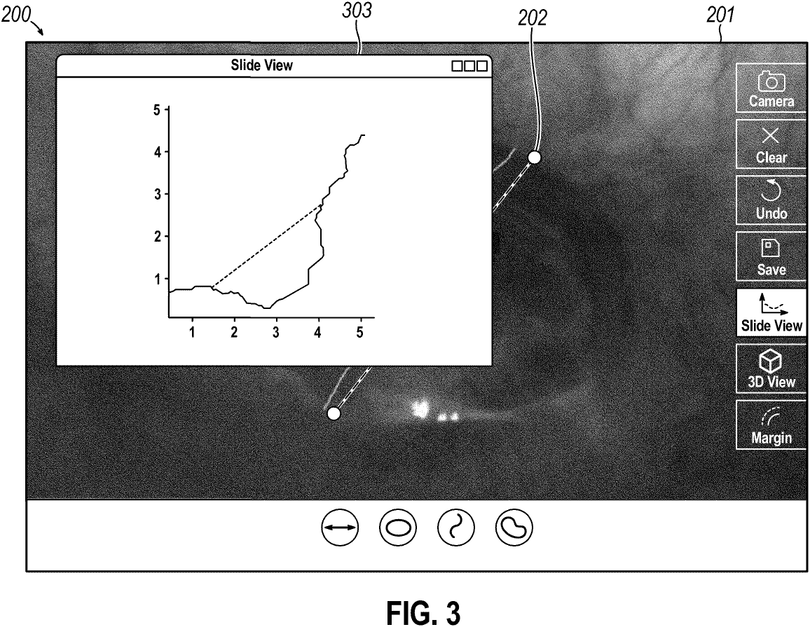

In another embodiment, the system may overlay various additional information, such as, for example, a 3D representation 203 of the area to be measured. Referring to FIG. 3, an additional embodiment is shown wherein a graphical representation 303 shows a cross section of the area to be measured, including various depth values and distances, overlayed on the surgical scene 201. Specifically, graph 303 represents a side-view of a point-to-point measurement of an in-vivo hernia defect. In some embodiments, and as shown, the graphical representation 303 may leverage the fundamental 3D reconstruction of the surgical scene from the acquired stereo images, in which a cutting-plane defining a side-view plane may be defined by the line between the selected points and the average scene surface plane (i.e., a plane including the line between the points selected by the user which is perpendicular to a plane which minimizes the squares of the distances from the surface of the surgical scene) and/or the camera’s direction axis (i.e., a plane including the line between the points selected by the user which is parallel to the camera’s direction axis).

Thus, the 3D surgical view 203 and/or cross-sectional views 303 may be used in some embodiments to help orient a user with the surface topography and associated measurements of the surgical scene to help a user ensure that a measurement reflects the proper points in a surgical scene. In a further embodiment, either display 203/303 may be rotated and/or zoomed to help a user view and\or understand how the selected points and measurements relate to the scene topography.

Moreover, as shown in FIGS. 2A and 2B, while user is selecting a point 202, that point may be rendered on both the 2D surgical scene 201 and the 3D surgical view 203 to help a user determine and confirm the exact location of the point. It should be understood that, although FIGS. 2A, 2B and 3 show the additional views 203/303 as an overlay, the views could also be shown separately (e.g., on separate devices). Referring to FIG. 1, a non-limiting example of this could be, for example, showing a 2D view one or more 2D monitors 104, while showing the 3D view on one or more 3D monitors 103. It is also possible that, rather than a surgeon monitor 103 showing a 3D view of information displayed in a 2D view on a touchscreen 104, the display of a touchscreen 104 may be duplicated on a surgeon view 103, such as using picture in picture, on a vertical split, in an overlaid window, or using such other simultaneous display mechanism as may be appropriate in a given case. In this way, a surgeon could continue to see real time information from the surgical scene (e.g., as captured through a laparoscope 102) while simultaneously seeing the touchscreen, allowing him or her to follow along with interactions (e.g., by an assistant) on the touchscreen 104 without displacing the view on the surgeon monitor 103 or requiring the surgeon himself or herself to directly interact with the touchscreen 104.

As shown in FIGS. 2A, 2B and 3, although the measurement input may be received relative to a 2D image, the actual calculations of the measurement may be performed on the underlying 3D scene 203 and/or the determined depth values 303. Thus, the distance measured may reflect the actual distance inside the surgical scene by taking camera pose (e.g., by using the camera direction to define the cutting plane) and scene topography into account. In some embodiments, the system may calculate the distance using direct or Euclidean method, in which a straight-line distance (e.g., between two points in three dimensional space) is calculated. In other embodiments, the system may follow the topography of the surgical scene when calculating a distance measurement. A further embodiment may allow for a user to select the method of calculation (e.g., in real time or through a user preference setting), or, alternatively, the system may automatically select the method based on various factors (e.g., what is being measured, the time required to calculate the measurement, the number of points being measured, etc.). Other embodiments may exist in which the system automatically calculates the distance in multiple ways simultaneously. In some such embodiments, the system may provide each calculated distance and its methodology to a user.

### II. Object Selection and Margin Generation

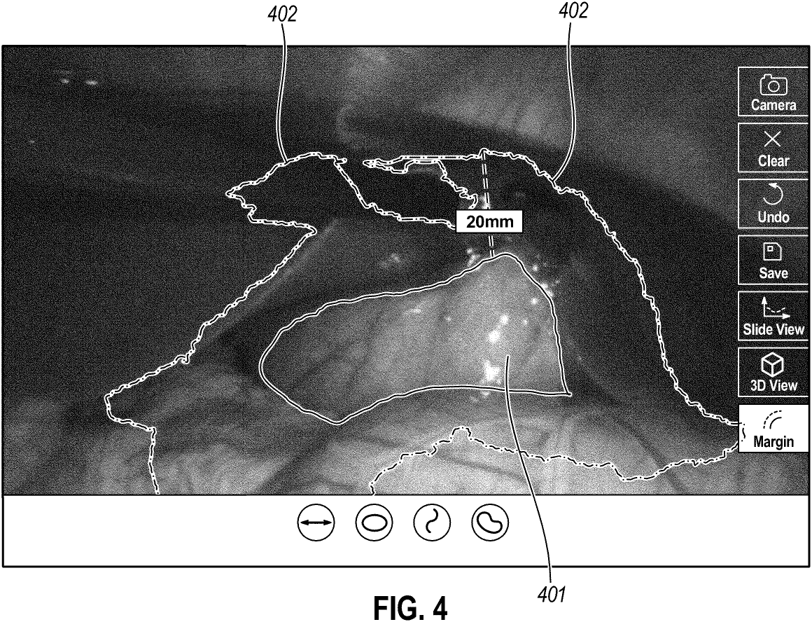

Referring now to FIG. 4, an example interface is provided with a particular object 401 (in the case of FIG. 4, a gallbladder, though the same approach could be applied to other objects, such as tumors or other parts of a patient’s anatomy) highlighted. In some cases, a system implemented based on this disclosure may allow a user to define the relevant object, such as by drawing a border around the object of interest. Alternatively, in some cases a system implemented based on this disclosure may allow a user to select a point on an object (e.g., a gallbladder), and may then automatically determine the boundaries of that object, such as through object recognition using computer vision, or multi-/hyperspectral imaging, such as described in U.S. Pat. Pub. No. 2020/0015925, entitled “Combination Emitter and Camera Assembly”, published Jan. 16, 2020, the disclosure of which is incorporated by reference herein in its entirety. As yet another alternative, in some cases a system implemented based on this disclosure may initially identify an object (e.g., using object recognition or hyperspectral imaging), and a user may be allowed to modify and/or adjust the boundary or other points or attributes of the object, such as using drawing tools in an interface as shown in FIG. 4.

As shown in FIG. 4, an interface such as may be shown by a system implemented based on this disclosure, in addition to showing a border of an object in a surgical scene 401, may also show a margin 402 around the border of the object. For example, a user may specify, as shown, a 20 mm margin should be shown that tracks the edge of the gallbladder 401 and extends along the surface of the patient’s body in three-dimensional space, which, as shown, can result in an irregularly shaped margin 402 when illustrated in a two-dimensional interface. In addition to, or as an alternative to, user specification, in some embodiments, the margin may be determined based on various factors, such as, for example, a surgical plan, one or more surgical tools being used, object recognition of the patient anatomy, and the like. Moreover, regardless of the method of implementation of the margin, in some embodiments, the system may, or may allow a user to, update and/or modify the margin (e.g., size, color, etc.).

In some embodiments, if a user specifies a margin which should be displayed around an object, a system implemented based on this disclosure may use a depth map of the surgical scene to automatically generate the margin and display it in an interface such as shown in FIG. 4. This may be used, for example, to illustrate a resection margin, which could be an area of tissue around a tumor (not shown) that appears to be non-tumorous tissue but may still be surgically removed for the safety of the patient. As noted, in some embodiments, the resection margin 402 may be user specified, while in other embodiments, the system may determine the resection size based on one or more known factors (e.g., a surgical plan, pre-operative images, user input, the results of the anatomy identification algorithm, or other known factors about the patient anatomy). Similar to the examples shown in FIGS. 2A, 2B and 3, FIG. 5 illustrates an embodiment in which a 3D representation 503 is shown overlayed on the surgical scene 501.

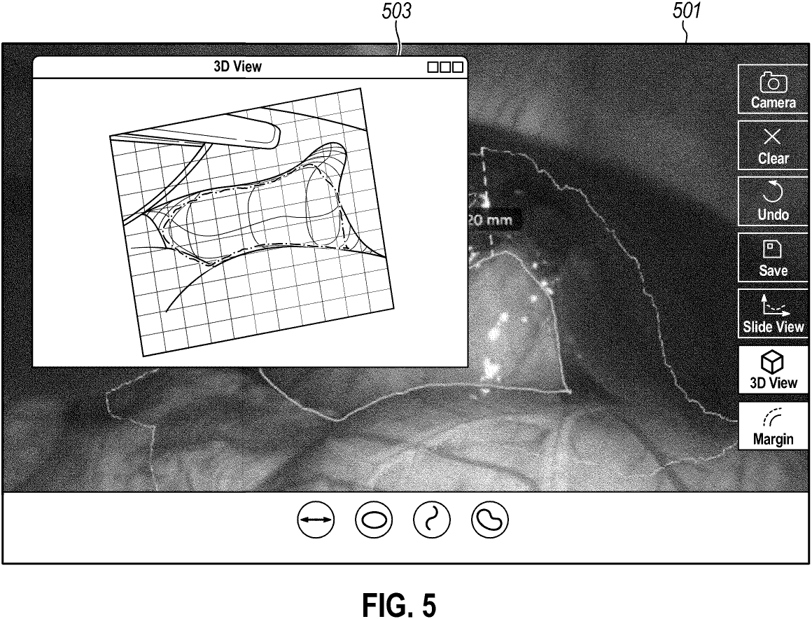

It should be understood that the above description of interfaces illustrating particular objects and methods of identifying those objects and their resection margins should be understood as being illustrative only, and that other variations are also possible. For example, in some cases, in addition to, or as an alternative to, highlighting the border of a specified object, a system implemented based on this disclosure may automatically calculate (e.g., using a depth map in a manner similar to that discussed in the context of FIGS. 2A and 2B) and display the longest measurement of the specified object. As another example, in some cases, the identification of a specified object may be facilitated by initially introducing fluorophores into the body of the patient and using the light from those fluorophores in the identification of the relevant object. Similarly, in some cases, an object in the interior of the cavity of a patient may initially be identified in a pre-operative image of the patient (e.g., a computed tomography or magnetic resonance imaging image), and then may be identified through registration of the pre-operative image with the real time image of the patient. Still further variations will be immediately apparent to and could be implemented without undue experimentation based on this disclosure by, one of ordinary skill in the art. Accordingly, the above description of variations, like the preceding discussion of FIGS. 4 and 5, should not be treated as implying limitations on the protection provided by this document or any other document which claims the benefit of this document.

### III. Tool Based Measurement

In addition to measuring points specified by a user and/or points that are specified automatically by the system, in some embodiments, the system may detect one or more tools within the surgical scene and determine a measurement utilizing their relative locations. Accordingly, in some embodiments, the system may analyze at least two images (e.g., a first image from a first image capture device and a second image from a second image capture device) in order to identify a first surgical tool and a second surgical tool. It should be understood that although the figures and disclosure generally refer to two tools, that the system can utilized more than two tools when conducting a tool based measurement.

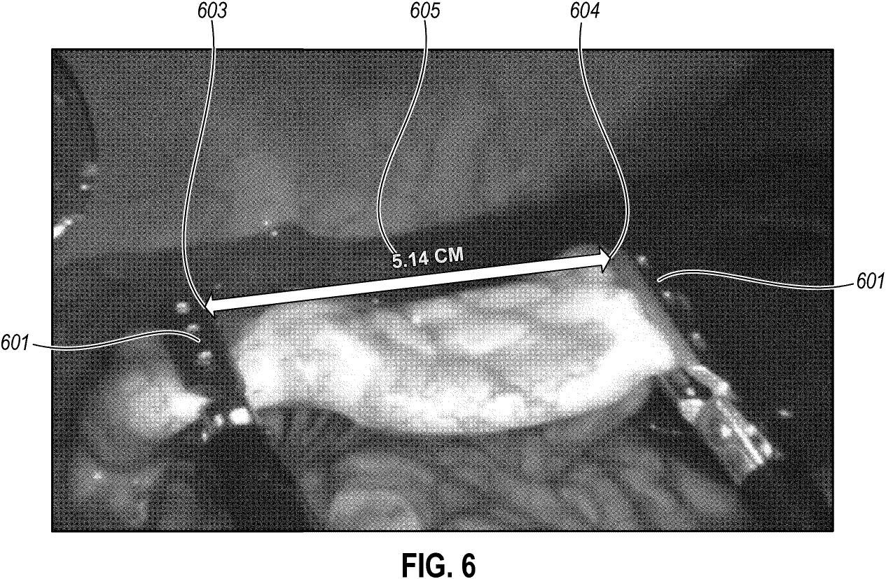

Referring now to FIG. 6, an example surgical scene 600 is shown including a first tool 601 and a second tool 602. In order for the system to detect the tools 601/602, computer vision is used to detect and determine the depth of the tool tips 603/604 (or other identifiable points on a tool, such as a pivot point of a tool, a handle, a tracking marker, a tip other than a distal tip, or any other identifiable point) of each tool. Specifically, the system may utilize a pair of stereo images, including two tools 601/602, and the calculated real-time 3D reconstruction of the surgical scene to identify both tool tips 603/604. In an alternative embodiment, the system may perform a direct triangulation of the tools (e.g., tool tips) 603/604. As should be understood by one of ordinary skill in the art, in an embodiment in which the system uses direct triangulation, a calculated 3D reconstruction of the surgical scene may not be required. Thus, in some embodiments, the system may be able to improve speed and reduce computations by performing a direct triangulation of the tools and their relative locations.

In order for the system to accurately identify the two tools 601/602 and determine the specified points to measure (e.g., the tool tips 603/604), the system may obtain or already have information associated with the specific tools. For example, the system may have been given a surgical plan that included a listing of all possible tools, and their specific characteristics, which would be used during the procedure. In an alternative example, the system may have, or obtain, a database of surgical tools, including their specific characteristics. Stated differently, the system my know, prior to analyzing the images, what tools are expected to be present in the surgical scene 600 and what their specific characteristics should be. Specifically, the system may know or obtain the tool size, shape, color, construction material, and the like. In a further embodiment, the tool may have an identifying marker that the system can use to associate it with a known tool’s characteristics. In an alternative embodiment, the system may use a deep-learning neural network that has been trained on various surgical tools to track and/or identify the tools.

Once the tool tips 603/604 are identified, the system uses their locations as the specified points and as discussed herein, calculates a measured distance between them. In some embodiments, the system may display (e.g., on a display device 103/104) a measurement value 605. Stated differently, in some embodiments, the system may analyze the stereo images to identify a first surgical tool and a second surgical tool and then determine a set of specified points based on a first point associated with the first surgical tool and a second point associated with the second surgical tool. A measurement is then calculated based on one of the methods disclosed herein (e.g., direct triangulation, using the depth map, and/or using a 3D reconstruction) and provided to the user (e.g., via display or audio device).

### IV. Temporally Extended Image Acquisition for Panoramic Reconstruction

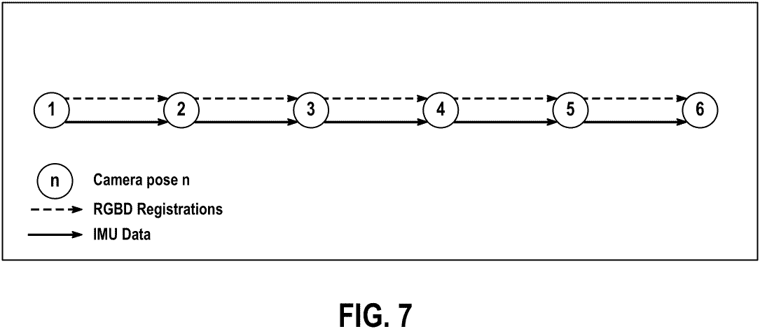

While the examples set forth herein may be implemented using 3D reconstructions generated based on combining left and right images from overlapping fields of view of an image sensor from a stereo camera, it should be understood that the disclosed technology may also be used in embodiments which provide reconstructions based on images extending beyond individual fields of view of a stereo camera’s image sensors, such as images captured over time as a stereo camera pans across a surgical scene. Examples of approaches which may be taken to support this type of panoramic reconstruction are described below in the context of FIGS. 7-9.

Turning first to FIG. 7, that figure provides a schematic illustration of potential relationships between multiple data sources (illustrated as RGBD registrations and IMU data) which could be used to generate a panoramic three-dimensional reconstruction. As would be understood by those of skill in the art, the fact that the surgical scene for minimally invasive surgery (MIS) is likely to be characterized by mucosal tissue, reflections, dark areas similar features can complicate the application of vision-based methods for image stitching and temporal reconstruction. Accordingly, in some cases, an imaging device (e.g., a laparoscopic camera) may be instrumented with an inertial measurement unit (IMU) such as could provide three axis measurements of linear acceleration and angular velocity while the imaging device is being used to capture images. This information may be used to determine how the imaging device’s pose (i.e., its position and orientation) changes between frames. This pose information may then, in turn, be used to determine how to stitch those frames together to perform a panoramic reconstruction of the surgical scene. However, as shown in FIG. 7, in some cases, rather than simply using pose information determined using IMU data, pose information may be determined using a combination of IMU data and visual data (labeled as RGBD, for red-green-blue-depth) from a stereo camera. In such a case, differences between RGBD images between frames may be used to determine transformation matrices representing pose changes between frames. The pose information determined from the RGBD and IMU data may then be combined (e.g., by averaging RGBD and IMU pose changes between frames) to obtain an estimated pose at each frame which may be more accurate than either the IMU or RGBD poses individually.

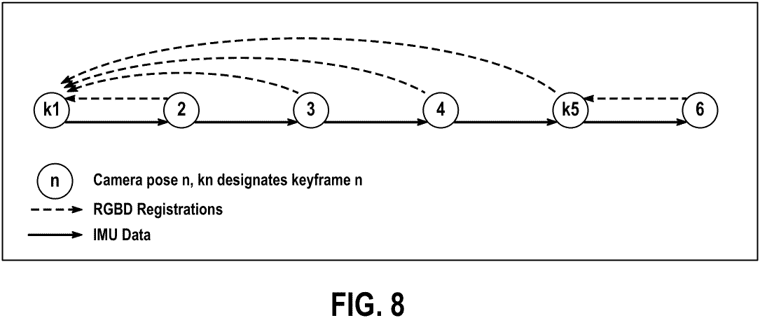

Other approaches to determining an imaging device’s pose by combining multiple types of data are also possible, and could be used in some embodiments which provide panoramic reconstruction functionality. An example of such an alternative approach is provided in FIG. 8, which illustrates an approach that uses RGBD data to generate poses based on differences between keyframes, rather than based on frame by frame differences as shown in FIG. 7. In particular, in an embodiment using an approach such as shown in FIG. 8, to determine the pose of a camera when a frame was captured, IMU data would be used in the same manner as described in the context of FIG. 7, while RGBD data would be used to determine the change between the camera’s position and orientation at that frame relative to the camera’s position and orientation at a most recent keyframe (indicated in FIG. 8 with the designation “k” prior to the frame number). The RGBD and IMU based pose information could then be combined as described in the context of FIG. 7, thereby providing a reconstruction which featured the increased accuracy of combining multiple types of data while also reducing the risk that frame-to-frame drift in RGBD data could function as a source of error.

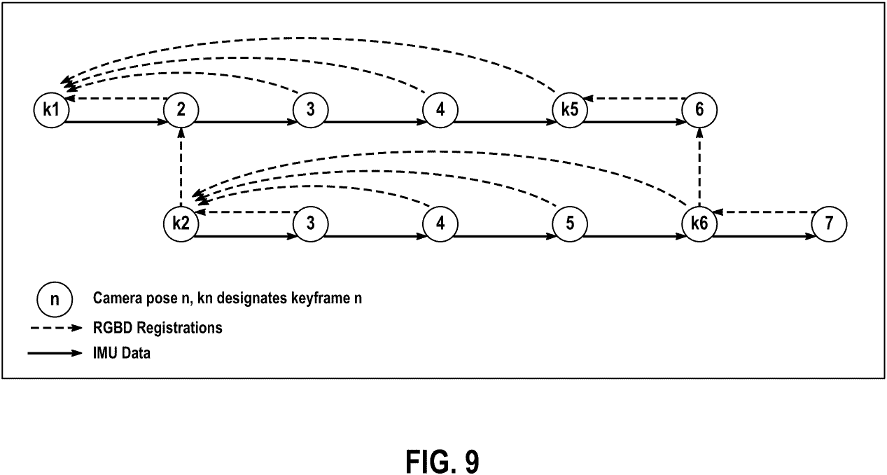

The keyframe based approach of FIG. 8 can also be extended to provide further stability for RGBD pose information. For example, as shown in FIG. 9, in some cases, a system implemented based on this disclosure may use parallel processing based on multiple keyframes to simultaneously generated multiple poses based on RGBD data. For example, a system implemented using the approach shown in FIG. 9 may generate a first RGBD pose for frame 3 by determining the movement of frame 3 relative to a first keyframe (shown in FIG. 9 as keyframe k1), and a second RGBD pose for frame 3 by determining the movement of frame 3 relative to a second keyframe (shown in FIG. 9 as keyframe k2). These different RGBD poses could then be combined (e.g., by averaging) to provide further stability before they were combined with a pose determined based on IMU data to generate a final pose that would be used for the panoramic reconstruction. This approach could also be extended to encompass additional parallel keyframe calculations, thereby allowing a system implemented based on this disclosure to provide additional stability if and as appropriate for a particular context. Further variations, even on this type of keyframe based approach (e.g., having keyframes every 15 frames rather than every 5 frames, dynamically determining keyframes when RGBD pose diverges from IMU pose by more than a threshold amount, etc.) are also possible, and will be immediately apparent to one of ordinary skill in the art in light of this disclosure. Accordingly, the above examples of potential approaches to combining multiple frames for panoramic image reconstruction should be understood as being illustrative only and should not be treated as limiting.

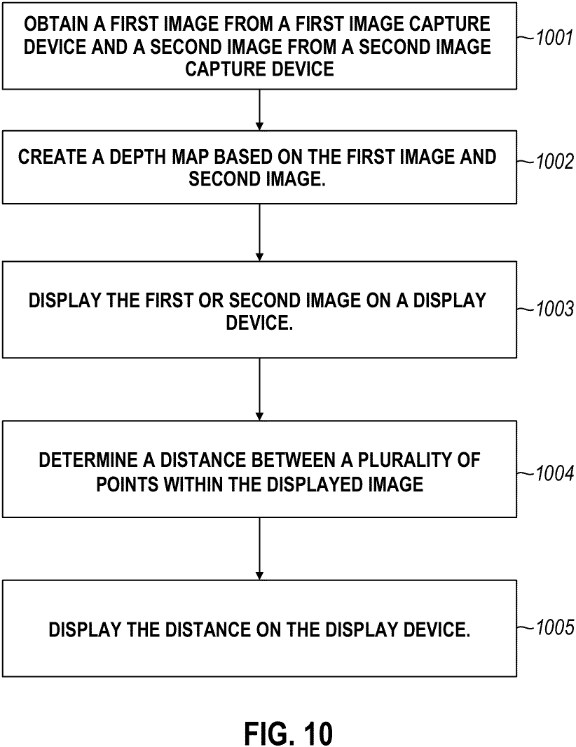

Whatever approaches are used to determine a 3D reconstruction, whether panoramic or otherwise, in some embodiments, such a reconstruction may be used in a method as illustrated in FIG. 10. As shown in FIG. 10, in some embodiments, the system may utilize a first image capture device and a second image capture device (potentially housed together) to capture first and second images of an interior of a cavity of a patient 1001. The system may then create, based on those images, a depth map 1002. During or after the creation of the depth map 1002, the system may display (e.g., on a display device such as 103/104) the first or second image 1003. The system may then identify a distance between a plurality of points 1004 (e.g., points selected by a user, determined algorithmically, or determined algorithmically based on user input, as described herein). Once the distance has been calculated, the system may then display the distance 1005. The system may also display on a display device, a three-dimensional view map of a portion of the interior of the cavity of the patient, a cross-sectional view of a portion of the interior of the cavity of the patient, or a graphical representation of the distance.

### V. Exemplary Combination

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system comprising: a) a first image capture device configured to capture a first image of an interior of a cavity of a patient; b) a second image capture device configured to capture a second image of the interior of the cavity of the patient; c) a two-dimensional display; d) a processor; and e) a non-transitory computer readable medium storing instructions operable to, when executed, cause the processor to perform a set of acts comprising: i) display, on the two-dimensional display, a two-dimensional image of the interior of the cavity of the patient; ii) determine a three-dimensional distance between a plurality of points on the two-dimensional image; and iii) display, on the two-dimensional display, the three-dimensional distance.

### Example 2

The system of example 1, wherein: a) the two-dimensional display is a touch display; b) the plurality of points on the two-dimensional image comprises a first point and a second point; and c) the non-transitory further stores instructions operable to, when executed, cause the processor to: i) receive the plurality of points on the two-dimensional image as user input provided by touching the touch display; ii) determine a three-dimensional location of each of the plurality of points using triangulation based on the first image and the second image; iii) determine the three-dimensional distance as the length of a straight line connecting, and having endpoints at, the first point and the second point in the three-dimensional space; iv) identify a cutting plane comprising the straight line connecting, and having endpoints at, the first point and the second point; and v) display, on the two-dimensional display simultaneously with the two-dimensional image of the interior of the cavity of the patient, a depiction of the straight line connecting, and having endpoints at, the first point and the second point in three-dimensional space on an image selected from a group consisting of: A) a cross-sectional view of a portion of the interior of the cavity of the patient taken on the cutting plane; and B) a three-dimensional reconstruction of the interior of the cavity of the patient which highlights a surface of the interior of the cavity of the patient intersecting the cutting plane.

### Example 3

The system of example 2, wherein the system comprises a laparoscope housing the first image capture device and the second image capture device, and wherein the cutting plane is selected from a group consisting of: a) a plane parallel to a direction of view of the laparoscope; and b) a plane perpendicular to a plane defined by an average surface of the cavity of the patient.

### Example 4

The system of example 1, wherein the two-dimensional image of the interior of the cavity of the patient is selected from a group consisting of: a) the first image of the interior of the cavity of the patient; b) the second image of the interior of the cavity of the patient; and c) a three-dimensional reconstruction of the interior of the cavity of the patient.

### Example 5

The system of example 1, wherein the instructions stored on the non-transitory computer readable medium are operable to, when executed, cause the processor to: a) create a depth map based on the first image and the second image; and b) determine, using the depth map, the three-dimensional distance between the plurality of points on the two-dimensional image as a distance between a first point and a second point from the plurality of points along a surface of the interior of the cavity of the patient on a plane comprising a straight line connecting the first point and the second point.

### Example 6

The system of example 1, wherein: a) the plurality of points on the two-dimensional image comprise: i) a point on a border of an anatomical object in the interior of the cavity of the patient; and ii) a point on an outer edge of a resection margin surrounding the anatomical object in the interior of the cavity of the patient; and b) the non-transitory computer readable medium further stores instructions operable to, when executed, cause the processor to: i) highlight the border of the anatomical object in the two-dimensional image of the interior of the cavity of the patient; and ii) highlight the outer edge of the resection margin surrounding the anatomical object in the two-dimensional image of the interior of the cavity of the patient.

### Example 7

The system of example 1, wherein: a) the system further comprises: i) a laparoscope housing the first image capture device; and ii) an inertial measurement unit (“IMU”) coupled to the laparoscope; and b) the non-transitory computer readable medium further stores instructions operable to, when executed, cause the processor to: i) generate a plurality of representations of the interior of the cavity of the patient, wherein each of the plurality of representations corresponds to a time from a plurality of times; and ii) for each time from the plurality of times, determine a pose corresponding to that time, based on: A) movement information captured from the IMU at that time; and B) the representation from the plurality of representations corresponding to that time; and iii) generate a panoramic view of the interior of the cavity of the patient based on combining the plurality of representations of the interior of the cavity of the patient using the poses corresponding to the times corresponding to those representations.

### Example 8

The system of example 7, wherein for each time from the plurality of times, determining the pose corresponding to that time comprises: a) determining a set of potential poses by, for each potential pose from the set of potential poses, determining that potential pose based on: i) the representation corresponding to that time, and ii) a different representation from the plurality of representations corresponding to a previous time; b) determining a representation pose corresponding to that time based on the set of potential poses; and c) determining the pose corresponding to that time based on: i) the representation pose corresponding to that time; and ii) an IMU pose based on the movement information captured from the IMU at that time.

### Example 9

The system of example 7, wherein: a) each representation from the plurality of representations is a three-dimensional representation of the interior of the cavity of the patient; and b) the non-transitory computer readable medium stores instructions operable to, when executed, cause the processor to generate each representation from the plurality of representations based on a pair of images captured by the first and second image capture devices at the corresponding time for that representation.

### Example 10

The system of example 1, wherein: a) the non-transitory computer readable medium further stores instructions operable to, when executed, cause the processor to: i) analyze the first image and the second image to identify a first surgical tool and a second surgical tool; and ii) determine, based on the analyzing, a first point associated with the first surgical tool and a second point associated with the second surgical tool; and b) the plurality of points on the two-dimensional image comprises the first point associated with the first surgical tool, and the second point associated with the second surgical tool.

### Example 11

The system of example 10, wherein the non-transitory computer readable medium further stores instructions operable to, when executed, cause the processor to display, on the two-dimensional display simultaneously with the two-dimensional image of the interior of the cavity of the patient, a depiction of a straight line connecting, and having endpoints at, the first point associated with the first surgical tool and the second point associated with the second surgical tool.

### Example 12

A method comprising: a) capturing a first image of an interior of a cavity of a patient and a second image of the interior of the cavity of the patient; b) displaying, on a two-dimensional display, a two-dimensional image of the interior of the cavity of the patient; c) determining a three-dimensional distance between a plurality of points on the two-dimensional image; and d) displaying, on the two-dimensional display, the three-dimensional distance.

### Example 13

The method of example 12, wherein: a) the two-dimensional display is a touch display; b) the plurality of points on the two-dimensional image comprises a first point and a second point; and c) the method further comprises: i) receiving the plurality of points on the two-dimensional image as user input provided by touching the touch display; ii) determining a three-dimensional location of each of the plurality of points using triangulation based on the first image and the second image; iii) determining the three-dimensional distance as the length of a straight line connecting, and having endpoints at, the first point and the second point in the three-dimensional space; iv) identifying a cutting plane comprising the straight line connecting, and having endpoints at, the first point and the second point; and v) displaying, on the two-dimensional display simultaneously with the two-dimensional image of the interior of the cavity of the patient, a depiction of the straight line connecting, and having endpoints at, the first point and the second point in three-dimensional space on an image selected from a group consisting of: A) a cross-sectional view of a portion of the interior of the cavity of the patient taken on the cutting plane; and B) a three-dimensional reconstruction of the interior of the cavity of the patient which highlights a surface of the interior of the cavity of the patient intersecting the cutting plane.

### Example 14

The method of example 13, wherein: a) the first and second images of the interior of the cavity of the patient are captured, respectively, by first and second image capture devices; b) the first and second image capture devices are housed within a laparoscope; c) the cutting plane is selected from a group consisting of: i) a plane parallel to a direction of view of a laparoscope housing first and second image; and ii) a plane perpendicular to a plane defined by an average surface of the cavity of the patient.

### Example 15

The method of example 12, wherein the two-dimensional image of the interior of the cavity of the patient is selected from a group consisting of: a) the first image of the interior of the cavity of the patient; b) the second image of the interior of the cavity of the patient; and c) a three-dimensional reconstruction of the interior of the cavity of the patient.

### Example 16

The method of example 12, wherein the method further comprises: a) creating a depth map based on the first image and the second image; and b) determining, using the depth map, the three-dimensional distance between the plurality of points as a distance between a first point and a second point from the plurality of points along a surface of the interior of the cavity of the patient on a plane comprising a straight line connecting the first pointe and the second point.

### Example 17

The method of example 12, wherein: a) the plurality of points on the two-dimensional image comprise: i) a point on a border of an anatomical object in the interior of the cavity of the patient; and ii) a point on an outer edge of a resection margin surrounding the anatomical object in the interior of the cavity of the patient; and b) the method further comprises: i) highlighting the border of the anatomical object in the two-dimensional image of the interior of the cavity of the patient; and ii) highlighting the outer edge of the resection margin surrounding the anatomical object in the two-dimensional image of the interior of the cavity of the patient.

### Example 18

The method of example 12, wherein the method further comprises: a) generating a plurality of representations of the interior of the cavity of the patient, wherein each of the plurality of representations corresponds to a time from a plurality of times; and b) for each time from the plurality of times, determining a pose corresponding to that time, based on: i) movement information captured from an inertial measurement unit (“IMU”) coupled to a laparoscope housing first and second image capture devices used to capture the first and second images of the interior of the cavity of the patient at that time; and ii) the representation from the plurality of representations corresponding to that time; and c) generating a panoramic view of the interior of the cavity of the patient based on combining the plurality of representations of the interior of the cavity of the patient using the poses corresponding to the times corresponding to those representations.

### Example 19

The method of example 18, wherein for each time from the plurality of times, determining the pose corresponding to that time comprises: a) determining a set of potential poses by, for each potential pose from the set of potential poses, determining that potential pose based on: i) the representation corresponding to that time, and ii) a different representation from the plurality of representations corresponding to a previous time; b) determining a representation pose corresponding to that time based on the set of potential poses; and c) determining the pose corresponding to that time based on: i) the representation pose corresponding to that time; and ii) an IMU pose based on the movement information captured from the IMU at that time.

### Example 20

The method of example 18, wherein the method comprises generating each representation from the plurality of representations based on a pair of images captured by the first and second image capture devices at the corresponding time for that representation.

### Example 21

The method of example 12, wherein: a) the method further comprises: i) analyzing the first image and the second image to identify a first surgical tool and a second surgical tool; and ii) determining, based on the analyzing, a first point associated with the first surgical tool and a second point associated with the second surgical tool; and b) the plurality of points on the two-dimensional image comprises the first point associated with the first surgical tool, and the second point associated with the second surgical tool.

### Example 22

The method of example 21, wherein the method further comprises displaying, on the two-dimensional display simultaneously with the two-dimensional image of the interior of the cavity of the patient, a depiction of a straight line connecting, and having endpoints at, the first point associated with the first surgical tool and the second point associated with the second surgical tool.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, which is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A system comprising:
a) a first image capture device configured to capture a first image of an interior of a cavity of a patient;
b) a second image capture device configured to capture a second image of the interior of the cavity of the patient;
c) a two-dimensional display;
d) a processor; and
e) a non-transitory computer readable medium storing instructions operable to, when executed, cause the processor to perform a set of acts comprising:
i) display, on the two-dimensional display, a two-dimensional image of the interior of the cavity of the patient;
ii) determine a three-dimensional distance between a plurality of points on the two-dimensional image; and
iii) display, on the two-dimensional display, the three-dimensional distance.

2. The system of claim 1, wherein:
a) the two-dimensional display is a touch display;
b) the plurality of points on the two-dimensional image comprises a first point and a second point; and
c) the non-transitory computer readable medium further stores instructions operable to, when executed, cause the processor to:
i) receive the plurality of points on the two-dimensional image as user input provided by touching the touch display;
ii) determine a three-dimensional location of each of the plurality of points using triangulation based on the first image and the second image;
iii) determine the three-dimensional distance as the length of a straight line connecting, and having endpoints at, the first point and the second point in the three-dimensional space;
iv) identify a cutting plane comprising the straight line connecting, and having endpoints at, the first point and the second point; and
v) display, on the two-dimensional display simultaneously with the two-dimensional image of the interior of the cavity of the patient, a depiction of the straight line connecting, and having endpoints at, the first point and the second point in three-dimensional space on an image selected from a group consisting of:
A) a cross-sectional view of a portion of the interior of the cavity of the patient taken on the cutting plane; and
B) a three-dimensional reconstruction of the interior of the cavity of the patient which highlights a surface of the interior of the cavity of the patient intersecting the cutting plane.

3. The system of claim 2, wherein the system comprises a laparoscope housing the first image capture device and the second image capture device, and wherein the cutting plane is selected from a group consisting of:
a) a plane parallel to a direction of view of the laparoscope; and
b) a plane perpendicular to a plane defined by an average surface of the cavity of the patient.

4. The system of claim 1, wherein the instructions stored on the non-transitory computer readable medium are operable to, when executed, cause the processor to:
a) create a depth map based on the first image and the second image; and
b) determine, using the depth map, the three-dimensional distance between the plurality of points on the two-dimensional image as a distance between a first point and a second point from the plurality of points along a surface of the interior of the cavity of the patient on a plane comprising a straight line connecting the first point and the second point.

5. The system of claim 1, wherein:
a) the plurality of points on the two-dimensional image comprise:
i) a point on a border of an anatomical object in the interior of the cavity of the patient; and
ii) a point on an outer edge of a resection margin surrounding the anatomical object in the interior of the cavity of the patient; and
b) the non-transitory computer readable medium further stores instructions operable to, when executed, cause the processor to:
i) highlight the border of the anatomical object in the two-dimensional image of the interior of the cavity of the patient; and
ii) highlight the outer edge of the resection margin surrounding the anatomical object in the two-dimensional image of the interior of the cavity of the patient.

6. The system of claim 1, wherein:
a) the system further comprises:
i) a laparoscope housing the first image capture device; and
ii) an inertial measurement unit (“IMU”) coupled to the laparoscope; and
b) the non-transitory computer readable medium further stores instructions operable to, when executed, cause the processor to:
i) generate a plurality of representations of the interior of the cavity of the patient, wherein each of the plurality of representations corresponds to a time from a plurality of times; and
ii) for each time from the plurality of times, determine a pose corresponding to that time, based on:
A) movement information captured from the IMU at that time; and
B) the representation from the plurality of representations corresponding to that time; and
iii) generate a panoramic view of the interior of the cavity of the patient based on combining the plurality of representations of the interior of the cavity of the patient using the poses corresponding to the times corresponding to those representations.

7. The system of claim 6, wherein for each time from the plurality of times, determining the pose corresponding to that time comprises:
a) determining a set of potential poses by, for each potential pose from the set of potential poses, determining that potential pose based on:
i) the representation corresponding to that time, and
ii) a different representation from the plurality of representations corresponding to a previous time;
b) determining a representation pose corresponding to that time based on the set of potential poses; and
c) determining the pose corresponding to that time based on:
i) the representation pose corresponding to that time; and
ii) an IMU pose based on the movement information captured from the IMU at that time.

8. The system of claim 6, wherein:
a) each representation from the plurality of representations is a three-dimensional representation of the interior of the cavity of the patient; and
b) the non-transitory computer readable medium stores instructions operable to, when executed, cause the processor to generate each representation from the plurality of representations based on a pair of images captured by the first and second image capture devices at the corresponding time for that representation.

9. The system of claim 1, wherein:
a) the non-transitory computer readable medium further stores instructions operable to, when executed, cause the processor to:
i) analyze the first image and the second image to identify a first surgical tool and a second surgical tool; and
ii) determine, based on the analyzing, a first point associated with the first surgical tool and a second point associated with the second surgical tool; and
b) the plurality of points on the two-dimensional image comprises the first point associated with the first surgical tool, and the second point associated with the second surgical tool.

10. The system of claim 9, wherein the non-transitory computer readable medium further stores instructions operable to, when executed, cause the processor to display, on the two-dimensional display simultaneously with the two-dimensional image of the interior of the cavity of the patient, a depiction of a straight line connecting, and having endpoints at, the first point associated with the first surgical tool and the second point associated with the second surgical tool.

11. A method comprising:
a) capturing a first image of an interior of a cavity of a patient and a second image of the interior of the cavity of the patient; depth map;
b) displaying, on a two-dimensional display, a two-dimensional image of the interior of the cavity of the patient;
c) determining a three-dimensional distance between a plurality of points on the two-dimensional image; and
d) displaying, on the two-dimensional display, the three-dimensional distance.

12. The method of claim 11, wherein:
a) the two-dimensional display is a touch display;
b) the plurality of points on the two-dimensional image comprises a first point and a second point; and
c) the method further comprises:
i) receiving the plurality of points on the two-dimensional image as user input provided by touching the touch display;
ii) determining a three-dimensional location of each of the plurality of points using triangulation based on the first image and the second image;
iii) determining the three-dimensional distance as the length of a straight line connecting, and having endpoints at, the first point and the second point in the three-dimensional space;
iv) identifying a cutting plane comprising the straight line connecting, and having endpoints at, the first point and the second point; and
v) displaying, on the two-dimensional display simultaneously with the two-dimensional image of the interior of the cavity of the patient, a depiction of the straight line connecting, and having endpoints at, the first point and the second point in three-dimensional space on an image selected from a group consisting of:
A) a cross-sectional view of a portion of the interior of the cavity of the patient taken on the cutting plane; and
B) a three-dimensional reconstruction of the interior of the cavity of the patient which highlights a surface of the interior of the cavity of the patient intersecting the cutting plane.

13. The method of claim 12, wherein:
a) the first and second images of the interior of the cavity of the patient are captured, respectively, by first and second image capture devices;
b) the first and second image capture devices are housed within a laparoscope;
c) the cutting plane is selected from a group consisting of:
i) a plane parallel to a direction of view of a laparoscope housing first and second image; and
ii) a plane perpendicular to a plane defined by an average surface of the cavity of the patient.

14. The method of claim 11, wherein the method further comprises:
a) creating a depth map based on the first image and the second image; and
b) determining, using the depth map, the three-dimensional distance between the plurality of points as a distance between a first point and a second point from the plurality of points along a surface of the interior of the cavity of the patient on a plane comprising a straight line connecting the first pointe and the second point.

15. The method of claim 11, wherein:
a) the plurality of points on the two-dimensional image comprise:
i) a point on a border of an anatomical object in the interior of the cavity of the patient; and
ii) a point on an outer edge of a resection margin surrounding the anatomical object in the interior of the cavity of the patient; and
b) the method further comprises:
i) highlighting the border of the anatomical object in the two-dimensional image of the interior of the cavity of the patient; and
ii) highlighting the outer edge of the resection margin surrounding the anatomical object in the two-dimensional image of the interior of the cavity of the patient.

16. The method of claim 11, wherein the method further comprises:
a) generating a plurality of representations of the interior of the cavity of the patient, wherein each of the plurality of representations corresponds to a time from a plurality of times; and
b) for each time from the plurality of times, determining a pose corresponding to that time, based on:
i) movement information captured from an inertial measurement unit (“IMU”) coupled to a laparoscope housing first and second image capture devices used to capture the first and second images of the interior of the cavity of the patient at that time; and
ii) the representation from the plurality of representations corresponding to that time; and
c) generating a panoramic view of the interior of the cavity of the patient based on combining the plurality of representations of the interior of the cavity of the patient using the poses corresponding to the times corresponding to those representations.

17. The method of claim 16, wherein for each time from the plurality of times, determining the pose corresponding to that time comprises:
a) determining a set of potential poses by, for each potential pose from the set of potential poses, determining that potential pose based on:
i) the representation corresponding to that time, and
ii) a different representation from the plurality of representations corresponding to a previous time;
b) determining a representation pose corresponding to that time based on the set of potential poses; and
c) determining the pose corresponding to that time based on:
i) the representation pose corresponding to that time; and
ii) an IMU pose based on the movement information captured from the IMU at that time.

18. The method of claim 16, wherein the method comprises generating each representation from the plurality of representations based on a pair of images captured by the first and second image capture devices at the corresponding time for that representation.

19. The method of claim 11, wherein:
a) the method further comprises:
i) analyzing the first image and the second image to identify a first surgical tool and a second surgical tool; and
ii) determining, based on the analyzing, a first point associated with the first surgical tool and a second point associated with the second surgical tool; and
b) the plurality of points on the two-dimensional image comprises the first point associated with the first surgical tool, and the second point associated with the second surgical tool.

20. The method of claim 19, wherein the method further comprises displaying, on the two-dimensional display simultaneously with the two-dimensional image of the interior of the cavity of the patient, a depiction of a straight line connecting, and having endpoints at, the first point associated with the first surgical tool and the second point associated with the second surgical tool.
